(19) United States
(12) Patent Application Publication
(10) Pub. No.: US 2003/0073926 A1
(43) Pub. Date: Apr. 17, 2003

(54) Method for setting volume and/or balance controls during a hearing test

(76) Inventors: Benny B. Johansen, Sunnyvale, CA (US); Ephram Cohen, San Francisco, CA (US); Sunil Puria, Mountain View, CA (US)

Correspondence Address: HAYNES BEFFEL & WOLFELD LLP, P O BOX 366, HALF MOON BAY, CA, 94019, US

(21) Appl. No.: 09/975,428

(22) Filed: Oct. 11, 2001

(51) Int. Cl. A61B 5/00

(52) U.S. Cl. 600/559; 600/300

(57) ABSTRACT

A method of testing the hearing of a user utilizing a computer system that includes a computer and a speaker. The computer can output an electrical signal to the speaker and the speaker is operable to convert the electrical signal into a stimulus. The computer system has volume controls that control the amplitude and balance between channels of the electrical signal. The method includes: downloading a computer program from a server to the computer; executing the computer program on the computer, the execution of the computer program setting a volume control; generating a stimulus; and receiving an input from the user that indicates that the user heard the stimulus.

This patent application discloses subject matter that is related to the subject matter disclosed in U.S. patent application Ser. No. __/______ entitled “Method For Setting Tone Controls During a Hearing Test,” __/______ entitled “Method For Muting and/or Un-Muting of Audio Sources During a Hearing Test,” and __/______ entitled “Method and System For Generating Audio Streams During a Hearing Test,” filed on even date herein. Each of the above Patent Applications is hereby incorporated by reference.

### FIELD OF THE INVENTION

The present invention generally relates to audiology and the presentation of audio signals for assessing a person's hearing using a personal computer. More specifically, the present invention relates to a system and a method for setting volume controls during a hearing test.

### BACKGROUND

Audiometric equipment exists for testing hearing. However, such equipment is expensive and is generally available only in hearing clinics. Many people are reluctant to visit hearing clinics and take a hearing test for a variety of reasons. Such reasons may include the cost of a hearing test, the time and inconvenience involved in scheduling of an appointment, waiting for and undergoing a hearing test, and privacy concerns. As a result, as many as 80 percent of the people who suffer from hearing loss in the United States may have not had their hearing tested.

Currently, a number of companies provide hearing tests over the Internet. For example, a user may have his/her hearing tested by accessing one of the following URLs: www.didyouhearme.com, www.handtronix.com, www.onlinehearing.com, www.audiainc.com, www.NigelWorks.com, www.audiologyawareness.com or www.freehearingtest.com.

For example, when a user accesses www.didyouheanne.com's hearing test, the user's computer system outputs a 500 Hz tone to a speaker, such as a powered or un-powered speaker or headphone that may include piezo electric transducers, which is coupled to the computer system. Next, the website instructs the user to decrease the volume on the user's computer until the user can no longer hear the 500 Hz tone.

There are several different volume controls that the user could utilize to decrease the volume on the user's computer system. In addition, there are several balance controls that the user could utilize to set the balance between the left and the right channel. For example, the user could adjust one or more volume and balance controls within the computer's operating system. For the purpose of the following discussion the term “volume control” will be used broadly for controls that adjust the overall level of sound or equivalent digital signals from one or more audio sources and/or adjust the balance between multiple channels of one or more audio sources. For example, many Windows operating systems include a Windows “Volume Control” applet window 100 such as shown in FIG. 1. This applet contains a number of volume controls 105, 106, 110, 111, 115, 116, 120, and 121. One volume control 105, a vertical slider bar in the “Wave” window of the applet, controls the amplitude of the electrical signals that are output to the user's computer system speakers when a user program sends a stream of digital amplitude values to the sound card driver. This stream may either be generated internally by the users program or read from an auxiliary file, such as a wave file and routed to the sound card driver audio source. Similarly a horizontal slider 106 controls the balance between the level output to the left and the right channel. Other volume controls 110 and 111, vertical and horizontal slider bars in the “MIDI” window, control the amplitude and channel balance of the electrical signals that are output to the speakers when a user program sends a stream of MIDI data to the sound card data. The stream of MIDI data may be generated internally by the user's program, read from an auxiliary file, such as a MIDI file or received from a connected MIDI source. Still other volume controls 115 and 116, vertical and horizontal slider bars in the “CD Audio” window, control the amplitude and channel balance of the electrical signals that are output to the speakers if the audio source is a compact disk (“CD”). The Windows “Volume Control” applet may also contain other volume controls that control the output amplitude of electrical signals from other audio sources such as “Microphone,” “Synthesizer,” “Line-In,” “Auxiliary” and “Modem.”

In addition to the above volume controls, other volume controls 120 and 121, vertical and horizontal slider bars in the “Volume Control” window, control the amplitude of the electrical signals that are output to the speakers regardless of the electrical signal's audio source.

Thus, if the source of the 500 Hz tone is a Wave file or an equivalent stream of digital data generated by the user's program, then the user could adjust the volume of the 500 Hz tone by adjusting the “Wave” window volume control 105 and/or the “Volume Control” window volume control 120.

In addition to adjusting the above volume controls, the user could also adjust a volume control on the computer speakers. As is well known, many speaker systems that are utilized with personal computers include a volume control.

As a result, the user has the option of adjusting at least three different volume controls to adjust the volume of the 500 Hz tone. It is possible for the user to set the volume of the 500 Hz tone so that the user can no longer hear the tone by setting the “Wave” window volume control 105, and/or the “Volume Control” window volume control 120 to a very low level and setting the speaker volume level to a very high level. In addition, setting the “Wave” window volume control 105, and/or the “Volume Control” window volume control 120 to a very high level and setting the speaker volume level to a very low level can set the volume of the 500 Hz tone to a similar level.

Next, the website generates a 4000 Hz tone. According to the website, if the user cannot hear the 4000 Hz tone, the user may have a hearing impairment. Next, the website generates a number of tones at different amplitudes at the following frequencies: 250, 500, 750, 1000, 1500, 2000, 3000, and 4000 Hz. When the user hears a particular frequency, the user presses an acknowledge button on the screen with the computer's mouse. Based upon the user's acknowledgements, the website generates an audiogram or equivalent measure for the user's hearing capabilities. An audiogram is a chart plotting a user's hearing threshold level in dB HL as a function of frequency. Using the audiogram, the user can determine if the user has a hearing impairment.

Unfortunately, setting the “Wave” window volume control 105, and/or the “Volume Control” window volume control 120 to either a very low level or a very high level can degrade the accuracy of the above hearing test. For example, if the volume controls 105 and/or 120 are set to a very low level, then the maximum sound amplitude achievable even when playing an audio stream with maximum possible digital values may be too low for the person to hear. In addition, if the volume controls 105 and/or 120 are set to a very high level, then the number of bits utilized by the computer system to output audio at a certain level will be reduced. It may be impossible to accurately generate signals at very low amplitude levels since the step-size at these levels will be too large. This may mean that a certain level cannot be generated accurately at all, or that it will be affected by quantization errors. Quantization errors can modify the spectral characteristics of the generated tones and can degrade the accuracy of the hearing test. Finally, if the output is not balanced between the multiple output channels (typically left and right), then the system may produce different sound levels for the left and right ear reducing the accuracy of the test.

Thus, a need exists for a hearing test that ensures the availability of the maximum required audio output level, and that avoids quantization and clipping errors.

### SUMMARY OF THE INVENTION

One embodiment of the invention is a method of testing the hearing of a user utilizing a computer system that includes a computer and a speaker. The computer can output an electrical signal to the speaker and the speaker is operable to convert the electrical signal into a stimulus. The computer system has a volume control that controls the amplitude of the electrical signal. The method includes: downloading a computer program from a server to the computer; executing the computer program on the computer, the execution of the computer program setting the volume control; generating a stimulus; and receiving an input from the user that indicates that the user heard the stimulus. In some embodiments the method also includes: sending data to the server; qualifying the hearing of the user; and sending other data to the computer.

Another embodiment of the invention is another method of testing the hearing of a user utilizing the above computer system. This method includes: downloading a computer program from a server to the computer; executing the computer program on the computer, the execution of the computer program storing the value of the volume control and setting the volume control; generating a stimulus; receiving an input from the user that indicates that the user heard the stimulus; and resetting the volume control to the stored value. In some embodiments the method also includes: sending data to the server; qualifying the hearing of the user; and sending other data to the computer.

Still other embodiments of the invention include program storage devices that contain computer readable instructions that, when executed, perform portions of the above methods.

### BRIEF DESCRIPTION OF THE FIGURES

FIG. 1 presents a “Volume Control” applet window.

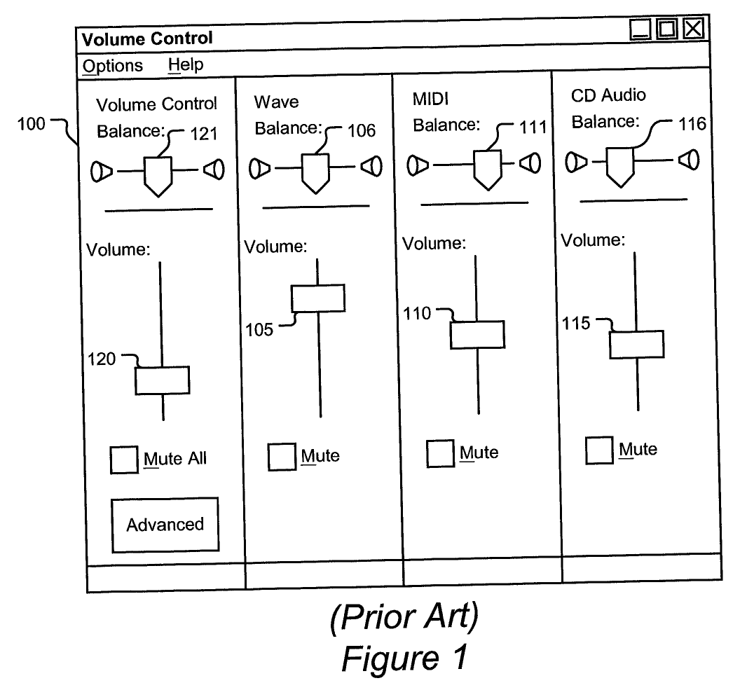

FIG. 2 presents a method of testing the hearing of a user that includes setting a volume control.

FIG. 3 presents another method of testing the hearing of a user that includes setting a volume control.

### DESCRIPTION OF THE PREFERRED EMBODIMENTS

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

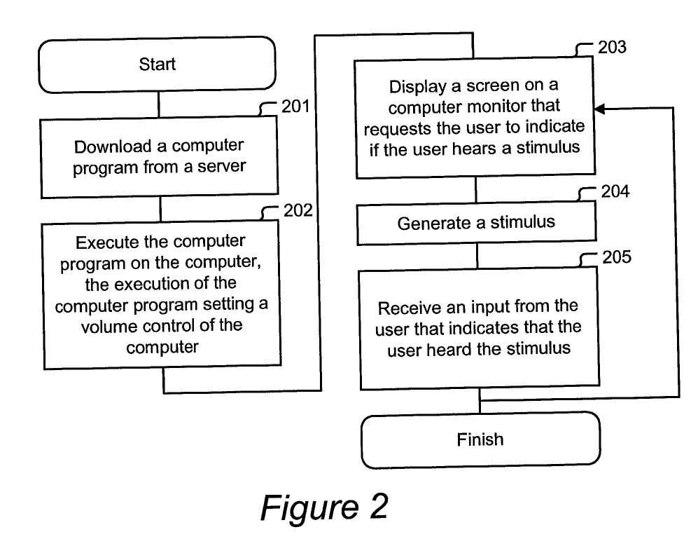

One embodiment of the invention, a method of testing the hearing of a user utilizing a computer system, is shown in FIG. 2. The method can be performed on a conventional computer system, such as a desktop computer system, a laptop computer system, or a handheld computer system. Other devices that include a microprocessor, such as a telephone, a mobile phone, a personal display assistant, an MP3 player, a radio, or a television, can also perform the method.

Hearing Tests that Set Volume Controls

Referring to Block 201 of FIG. 2, a user that desires to take a hearing test first downloads a computer program, such as a stand-alone executable program, a Java applet, an Active X control, or a Netscape plugin, from another computer, such as a server, to his/her computer. In one embodiment of the invention, the computer program is transferred via the Internet. In another embodiment of the invention, the computer program is transferred via an email. As is well known, computer programs may be attached to emails that can be easily distributed over the Internet, virtual private networks, local area networks and/or wide area networks. In still other embodiments, the computer program could be transferred to the user via the United States postal service or other postal service.

Next, referring to Block 202 of FIG. 2, the user executes the computer program on the user's computer. The execution of the computer program sets a volume control, such as the above-described “Volume Control” volume control, to a predefined volume level such as 50%. In some embodiments of the invention, the computer program would also set the “Wave” volume control to 50%. If the user needs to adjust the volume of the sound output by the speaker, then the user can adjust the volume by setting the speaker volume.

Next, referring to Block 203 of FIG. 2, the computer program displays a screen on the computer monitor that requests the user to indicate if the user hears a stimulus. Then, referring to Block 204 of FIG. 2, the computer program outputs a stimulus. If the user hears the stimulus, then the user inputs information, such as via a mouse click or a keyboard entry, into the computer that indicates that the user hears the stimulus. Referring to Block 205 of FIG. 2, the computer program then receives the input from the user that indicates whether or not the user heard the stimulus.

As shown in FIG. 2, by repeating Blocks 203 through 205 with stimuli of different amplitudes and frequencies, data sufficient to quantify the hearing of the user can be derived using conventional methods. In some embodiments of the invention, the computer program qualifies the hearing. In other embodiments, the computer program transfers data to a server and the server qualifies the hearing and then sends data back to the computer program. After the hearing of the user is quantified, some embodiments of the invention present an audiogram, text information, and/or graphical information to the user.

By setting one or more volume controls to a predefined volume level, the accuracy of the hearing test may be increased. Further the range of audio levels, which can be produced without introducing excessive quantization is increased, leading to a more accurate test. Setting these levels automatically relieves the user of potentially having to do this, leading to a faster and more user friendly test. Finally, the volume and balance controls may be set more accurately by the program than a user would be able to do by manually moving a slider.

Other Embodiments of the Invention

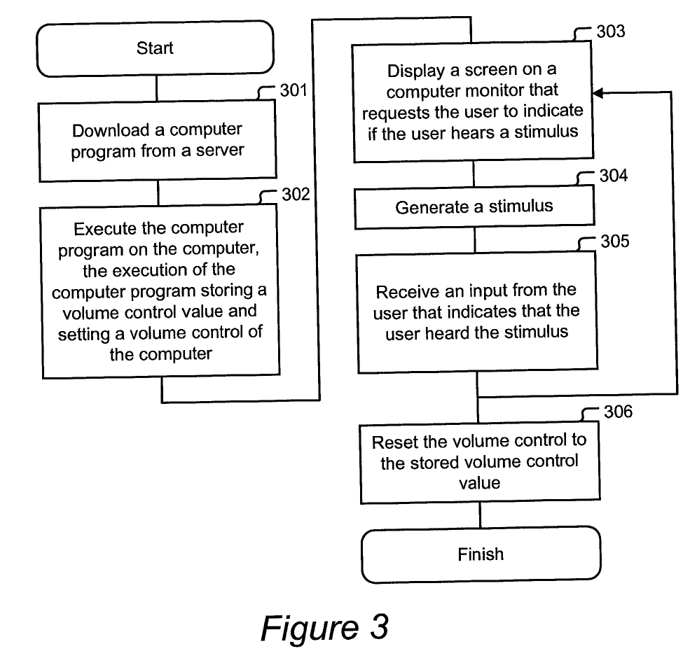

If the user had previously set one or more volume controls, then the above method would “overwrite” the previously set volume control value(s). Thus, in some embodiments of the invention, such as shown in FIG. 3, the computer program would store one or more previously set volume control values before setting the volume controls. Then, after the conclusion of the hearing test, the computer program would reset the volume controls to the previously set values.

Conclusion

The foregoing descriptions of embodiments of the present invention have been presented for purposes of illustration and description only. They are not intended to be exhaustive or to limit the present invention to the forms disclosed. Accordingly, many modifications and variations will be apparent to practitioners skilled in the art. For example, program storage devices, such as hard disks, floppy disks, random access memories (RAM), read only memories (ROM), programmable read only memories (PROM), compact disks (CD), and digital versatile disks that contain computer readable instructions that perform portions of the above methods, are intended to be included in the present invention. Additionally, the above disclosure is not intended to limit the present invention. The scope of the present invention is defined by the appended claims.

It is claimed:

1. A method of testing the hearing of a user utilizing a computer system, the computer system including a computer and a speaker, the computer operable to output an electrical signal to the speaker, the speaker operable to convert the electrical signal into a stimulus, the computer system having a volume control that controls the amplitude of the electrical signal, the method comprising:
a) downloading a computer program from a server to the computer;
b) executing the computer program on the computer, the execution of the computer program setting the volume control;
c) generating a stimulus; and
d) receiving an input from the user that indicates whether or not the user heard the stimulus.

2. The method of claim 1, wherein the act of downloading the computer program includes transferring the computer program from the server to the computer via the Internet.

3. The method of claim 1, wherein the act of downloading the computer program includes transferring the computer program from the server to the computer via an email.

4. The method of claim 1, wherein the act of executing the computer program includes setting a volume control that controls the amplitude of electrical signals from a single audio source.

5. The method of claim 1, wherein the act of executing the computer program includes setting a volume control that controls the channel balance between electrical signals from a single audio source.

6. The method of claim 1, wherein the act of executing the computer program includes setting a volume control that controls the amplitude of electrical signals from a Wave source.

7. The method of claim 1, wherein the act of executing the computer program includes setting a volume control that controls the amplitude of electrical signals from a stream of digital audio data generated by the computer program.

8. The method of claim 1, wherein the act of executing the computer program includes setting a volume control that controls the amplitude of electrical signals from a plurality of audio sources.

9. The method of claim 1, wherein the act of executing the computer program includes setting a volume control that controls the channel balance of electrical signals from a plurality of audio sources.

10. The method of claim 1, wherein the act of executing the computer program includes setting a first volume control that controls the amplitude of electrical signals from a single audio source and setting a second volume control that controls the amplitude of electrical signals from a plurality of audio sources.

11. The method of claim 1, wherein the act of executing the computer program includes setting a first volume control that controls the channel balance of electrical signals from a single audio source and setting a second volume control that controls the channel balance of electrical signals from a plurality of audio sources.

12. The method of claim 1, further including:
a) sending first data to the server;
b) qualifying the hearing of the user; and
c) sending second data to the computer.

13. A method of testing the hearing of a user utilizing a computer system, the computer system including a computer and a speaker, the computer operable to output an electrical signal to the speaker, the speaker operable to convert the electrical signal into a stimulus, the computer system having a volume control that controls the amplitude of the electrical signal, the method comprising:
a) downloading a computer program from a server to the computer;
b) executing the computer program on the computer, the execution of the computer program storing a value of the volume control and setting the volume control;
c) generating a stimulus;
d) receiving an input from the user that indicates whether or not the user heard the stimulus; and
e) resetting the volume control to the stored values.

14. The method of claim 13, wherein the act of downloading the computer program includes transferring the computer program from the server to the computer via the Internet.

15. The method of claim 13, wherein the act of downloading the computer program includes transferring the computer program from the server to the computer via an email.

16. The method of claim 13, wherein the act of executing the computer program includes storing the value of a volume control that controls the amplitude of electrical signals from a single audio source and setting a volume control that controls the amplitude of electrical signals from a single audio source.

17. The method of claim 13, wherein the act of executing the computer program includes storing the value of a volume control that controls the amplitude of electrical signals from a Wave audio source and setting a volume control that controls the amplitude of electrical signals from a Wave audio source.

18. The method of claim 13, wherein the act of executing the computer program includes storing the value of a volume control that controls the amplitude of electrical signals from a Wave audio source and setting a volume control that controls the amplitude of electrical signals from a stream of digital audio data that was generated within the computer program.

19. The method of claim 13, wherein the act of executing the computer program includes storing the value of a volume control that controls the amplitude of electrical signals from a plurality of audio sources and setting a volume control that controls the amplitude of electrical signals from a plurality of audio sources.

20. The method of claim 13, wherein the act of executing the computer program includes storing the value of a first volume control that controls the amplitude of electrical signals from a single audio source, storing the value of a second volume control that controls the amplitude of electrical signals from a plurality of audio sources, setting a first volume control that controls the amplitude of electrical signals from a single audio source, and setting a second volume control that controls the amplitude of electrical signals from a plurality of audio sources.

21. The method of claim 13, further including:
a) sending first data to the server;
b) qualifying the hearing of the user; and
c) sending second data to the computer.

22. A program storage device that contains computer readable instructions that, when executed by a computer system having a volume control, tests the hearing of a user by:
a) setting the volume control of the computer;
b) generating a stimulus; and
c) receiving an input from the user that indicates that the user heard the stimulus.

23. The program storage device of claim 22, wherein the act of setting the volume control includes setting a volume control that controls the amplitude of electrical signals from a Wave audio source.

24. The program storage device of claim 22, wherein the act of setting the volume control includes setting a volume control that controls the amplitude of electrical signals from a stream of digital audio data generated within the computer program.

25. The program storage device of claim 22, wherein the act of setting the volume control includes setting a volume control that controls the amplitude of electrical signals from a plurality of audio sources.

26. The program storage device of claim 22, wherein the act of setting the volume control includes setting a first volume control that controls the amplitude of electrical signals from a single audio source and setting a second volume control that controls the amplitude of electrical signals from a plurality of audio sources.

27. A program storage device that contains computer readable instructions that, when executed by a computer system having a volume control, tests the hearing of a user by:
a) storing the value of the volume control
b) setting the volume control;
c) generating a stimulus;
d) receiving an input from the user that indicates whether or not the user heard the stimulus; and
e) resetting the volume control to the stored value.

28. The program storage device of claim 27, wherein the act of storing the value of the volume control includes storing the value of a volume control that controls the amplitude of electrical signals from a single audio source.

29. The program storage device of claim 27, wherein the act of storing the value of the volume control includes storing the value of a volume control that controls the amplitude of electrical signals from a Wave audio source.

30. The program storage device of claim 27, wherein the act of storing the value of the volume control includes storing the value of a volume control that controls the amplitude of electrical signals from a stream of digital audio data generated within the computer program.

31. The program storage device of claim 27, wherein the act of storing the value of the volume control includes storing the value of a volume control that controls the amplitude of electrical signals from a plurality of audio sources.

32. The program storage device of claim 27, wherein the act of storing the value of the volume control includes storing the value of a first volume control that controls the amplitude of electrical signals from a single audio source and storing the value of a second volume control that controls the amplitude of electrical signals from a plurality of audio sources.
